(19) United States
(12) Patent Application Publication
(10) Pub. No.: US 2007/0078502 A1
(43) Pub. Date: Apr. 5, 2007

(54) METHOD AND APPARATUS FOR ESTIMATING A LOCAL IMPEDANCE FACTOR

(75) Inventors: Bryan Weber, Livermore, CA (US); Karl Pope, San Mateo, CA (US); Mitchell Levinson, Pleasanton, CA (US)

Correspondence Address: WOOD, HERRON & EVANS, LLP, 2700 CAREW TOWER, 441 VINE STREET, CINCINNATI, OH, 45202, US

(73) Assignee: THERMAGE, INC., Hayward, CA (US)

(21) Appl. No.: 11/470,041

(22) Filed: Sep. 5, 2006

Related U.S. Application Data
(60) Provisional application No. 60/723,695, filed on Oct. 5, 2005.

(51) Int. Cl. A61F 2/00

(52) U.S. Cl. 607/101; 600/547

(57) ABSTRACT

Method and apparatus for determining local impedance factors in an electromagnetic energy system for treating patients is disclosed. Respective measurement signals are sent through a patient treatment zone and a local impedance factor is estimated based upon the measurement signals. The estimated impedance factor is used to determine appropriate therapeutic levels of energy for patient treatment.

### CROSS-REFERENCE TO RELATED APPLICATIONS

This application claims the benefit of U.S. Provisional Application Ser. No. 60/723,695 filed Oct. 5, 2005, the disclosure of which is hereby incorporated by reference herein in its entirety.

### FIELD OF THE INVENTION

The invention relates to method and apparatus for estimating local impedance factors. More particularly, the invention relates to method and apparatus for determining a local impedance factor in an electromagnetic energy delivery device used to non-invasively treat patients.

### BACKGROUND OF THE INVENTION

Electromagnetic energy delivery devices are often utilized to treat patients for various medical, cosmetic, and therapeutic reasons. For example, such devices may be utilized to heat tissue to within a selected temperature range to produce a desired effect, such as improving the appearance of the patient by removing or reducing wrinkles, tightening skin, removing hair, etc. Such devices generate a signal, such as an optical, infrared, microwave, or radiofrequency (RF) signal, which is then applied to the patient to heat tissue in a desired manner. Examples of such electromagnetic energy delivery devices are disclosed in commonly-assigned U.S. Pat. Nos. 5,660,836 and 6,350,276, the disclosure of each of which is incorporated by reference herein in its entirety.

Because of the energy associated with these signals and their application to human patients, generation and use of these systems must be controlled to ensure that sufficient energy is applied to achieve an adequate therapeutic effect without harming the patient. In order to monitor and control the application of energy, various radiofrequency devices sense applied currents and voltages to ensure that these parameters are within pre-determined operational ranges. These devices also measure or calculate total system impedance, and control voltages and currents consistent with the impedance measured so that only predetermined ranges of radiofrequency energies are delivered by the device to the patient. However, merely sensing applied currents and voltages and delivering controlled amounts of energy is not necessarily indicative of an appropriate level of treatment being applied to each patient because each patient generally presents varying physical properties that are uniquely effected by the applied energy.

For example, part of the energy delivered by the device is absorbed by a patient treatment zone to heat this zone. Another part of the delivered energy is absorbed by the patient in locations remote from the treatment zone, which results in non-therapeutic heating in these removed locations. Still further parts of the delivered energy are absorbed by the device delivery and return wires, connectors, and other components. The distribution of the energy absorption varies from treatment zone to treatment zone for any given patient, and varies among different patients. As a specific numerical example, if the intent is to deliver 50 joules of energy to a treatment zone, a device energy delivery setting of 150 joules will be suffice when a local impedance of the treatment zone is one third of the total system impedance. When the local impedance of the treatment zone is larger, excessive energy may be delivered to the treatment zone, which may damage the tissue. Conversely, when the local impedance of the treatment zone is smaller, insufficient energy may be delivered to the treatment zone so that the desired therapeutic result (e.g., tissue tightening) is not achieved.

The determination of the fraction and, hence, amount of energy absorbed by a localized patient treatment zone requires some knowledge of the local impedance associated with the treatment zone and other device and system impedances. Because these impedances vary from patient-to-patient and are non-constant for different treatment areas on any given patient, a clinician may rely on patient pain feedback to properly set the energy delivery settings on these devices to deliver a therapeutic amount of energy to the treatment zone. Patient feedback is described in U.S. Publication No. 20030236487, the disclosure of which is incorporated by reference herein in its entirety. Reliance on patient feedback is disadvantageous because, on one hand, the amount of energy delivered to a patient with a low tolerance for pain may be non-therapeutic. On the other hand, excessive energy may be delivered to a patient that is overly pain-tolerant based on the lack of a verbalized pain feedback.

Therefore, an apparatus and method are needed for estimating impedances associated with patient treatment zones and other device and system impedances so that appropriate energy levels may be delivered to the patient treatment zone to achieve therapeutic results and yet not harm a patient.

### SUMMARY OF THE INVENTION

The invention overcomes the problems outlined above, as well as other problems with conventional treatment methods and devices, and provides improved methods and electromagnetic energy devices for the treatment of patients at specific patient treatment zones. In order to better determine appropriate therapeutic energy levels, the invention estimates local impedance factors associated with respective patient treatment zones, and uses these estimated factors to determine the energy levels. Generally, the methods of patient treatment of the invention comprise sending first and second measurement signals partially through a patient treatment zone to determine corresponding measurement values and using the determined measurement values to estimate the local impedance factor.

In embodiments of the invention, the first and second measurement values may be determined by measuring at least one parameter selected from the group consisting of currents and voltages associated with the first and second measurement signals. Advantageously, the estimated local impedance factor may be determined as a ratio between the local impedance associated with the treatment zone and a total system impedance of the device.

The electrical impedance is a complex number characterized by a resistance R, which comprises the real part of the complex number, and a capacitive reactance, which comprises the imaginary part of the complex number. The first and second measurement values used to estimate the local impedance factor may reflect the voltage, current, impedance, and phase angle relationship between the voltage and current of the measurement signal, as understood by a person having ordinary skill in the art.

In actual treatment practice, a patient is typically treated by repetitively sending respective therapeutic signals through individual treatment zones. In such a case, individual estimated local impedance factors are determined for each of these treatment zones, and the corresponding magnitudes of therapeutic energy are determined using such local impedance factors.

Improved electromagnetic energy patient treating devices include an electromagnetic energy generator and a treatment tip operatively coupled with the generator to deliver electromagnetic energy into patient treatment zones. The generator includes a controller, which may be housed with the energy generator or separate therefrom, for delivering at least first and second measurement signals to the tip for passage into the patient treatment zone. The controller is operable to estimate the local impedance factor associated with the patient treatment zone using data derived from the measurement signals.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1a is a schematic view of a system or device constructed in accordance with the principles of various embodiments of the invention, as applied to a patient undergoing treatment.

FIG. 1b is a schematic view showing energy transmission characteristics of the system of FIG. 1a.

FIG. 1c is a simplified electrical schematic of the system of the system of FIG. 1a.

FIG. 1d is an electrical schematic associated with an embodiment of the invention.

FIG. 1e is another electrical schematic pertinent to an embodiment of the invention.

FIG. 2 is a schematic view of a system in accordance with an alternative embodiment of the invention in which a measurement electrode is separate from a treatment electrode.

FIG. 3 is a schematic view of an embodiment of the invention that utilizes a treatment electrode that also functions as a measurement electrode.

FIG. 4a is a perspective view of the treatment/measurement electrode of FIG. 3.

FIG. 4b is a graph illustrating an interpolation estimation technique associated with an embodiment of the invention.

FIG. 4c illustrates another embodiment of a measurement/treatment electrode of the invention.

FIG. 5 is a block diagram of the system of an embodiment of the invention illustrating various system elements.

### DETAILED DESCRIPTION

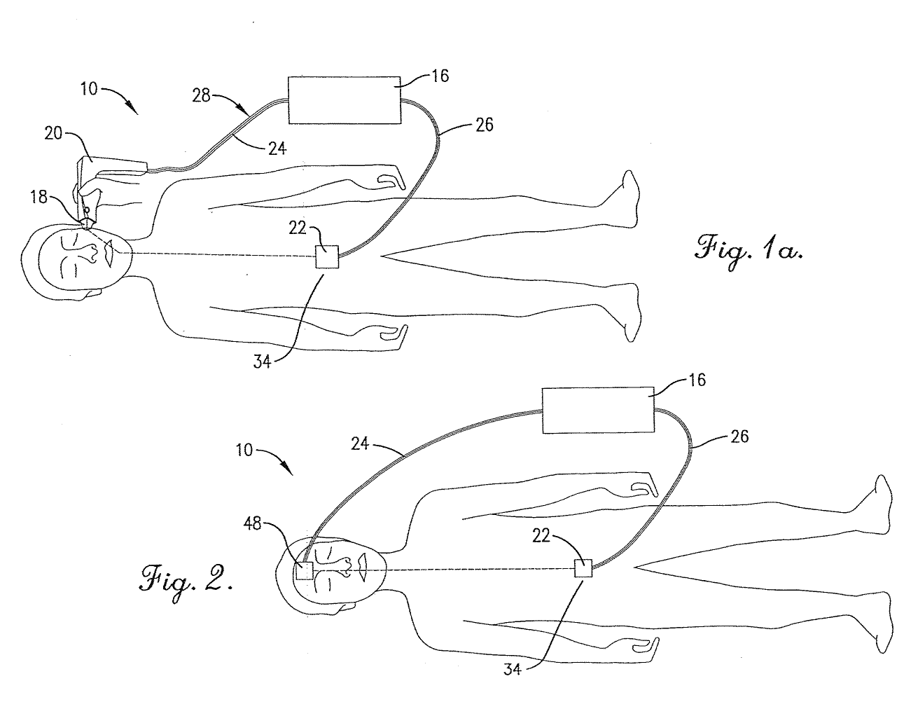

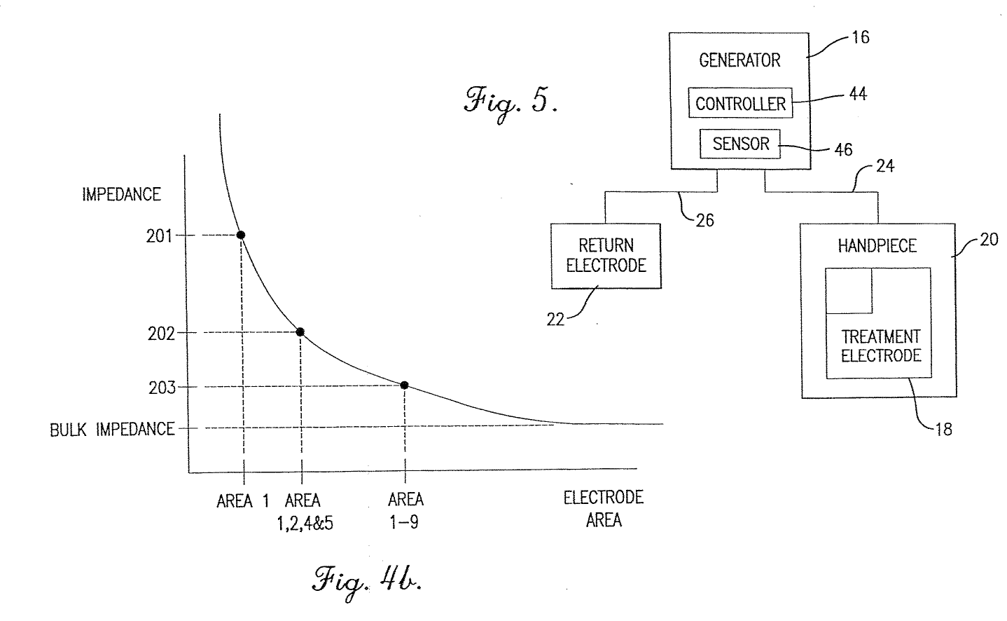

With reference to FIGS. 1a, 1b, and 5, a system 1O generally includes a generator 16 for generating a treatment signal and one or more measurement signals, a treatment electrode 18 which may be mounted on handpiece 20, and a second return electrode 22. The electrodes 18, 22 are coupled with the generator 16 via cables 24, 26, respectively. System 10 may be used to perform any therapeutic, medical, and/or cosmetic-related treatment for which it is suited.

Generator 16 may include other elements in addition to the signal generation elements, such as a controller 44 and at least one sensor 46. Sensor 46 may detect any one of any of signal current, voltage, resistance, impedance, and/or other signal parameters. The controller 44 and sensor 46 may be integral within the same housing as other elements of the generator 16, such as within a common generator housing, or the controller 44, sensor 46, and other generator elements, such as the signal generating elements, may be positioned within separate housings, e.g., multiple housings or units. Generator 16 is operable to generate a signal, such as a radio frequency or microwave signal, utilizing generally known and conventional signal generation elements. The generator 16 may be operable to generate a high frequency signal, such as a radiofrequency signal having a frequency in the range of about 1 MHz to about 20 MHz.

In use, the generator 16 generates a treatment signal 28 that flows through generator cable 24, into treatment handpiece 20, through treatment electrode 18, through patient skin surface 14, and through treatment zone 12. The portion of the treatment electrode 18 contacting the skin surface 14 may be cooled during generation and transfer of the treatment signal 28. The treatment signal 28 then flows through body tissue 30 that is outside the zone 12, through remote patient tissue zone 32 and skin surface 34, through return electrode 22, and finally through generator return cable 26.

When the treatment signal 28 is a radiofrequency signal, the energy associated with the signal may be represented by electromagnetic field vectors, as indicated diagrammatically by the radiating lines 36 in FIG. 1b. To promote therapeutic heating of the tissue in the treatment zone 12, the electrode 18 is sized sufficiently small so that the field 36 in the vicinity of the electrode 18 is concentrated. Heat is deposited at a rate in zone 12 that far exceeds the heat removal capacity of tissue in the zone 12, which results in a therapeutic rise in tissue temperature as energy is delivered. At some distance 38 remote from the electrode 18, the field is diffused to such an extent that the density of deposited heat is diminished. As a result, therapeutic heating does not occur beyond distance 38 from the electrode 18. This is because of the quantity of deposited heat is insufficient to raise local temperatures in this deeper tissue zone to a therapeutic level and because of the heat removal capacity of tissue in this deeper tissue zone.

Treatment electrodes 18 that capacitively couple energy with tissue in the treatment zone 12 may be as small as about 0.10 cm² to about 20 cm² and still result in therapeutic heating of zone 12. The treatment zone 12 may have a depth of about 1 mm to about 40 mm, depending on the amount and rate of energy delivery and other system and physiological parameters. Therapeutic electrodes having areas in the range of about 0.25 cm² to about 10 cm² are quite typical. Capacitively coupled treatment electrodes 18 suitable for use in the invention are described in U.S. Pat. No. 6,413,255, the disclosure of which is incorporated by reference herein in its entirety.

Conversely, therapeutic heating in zone 32 adjacent the return electrode 22 is generally not desired, particularly for an RF monopolar system represenative of an embodiment of the invention. Such heating is prevented by making the return electrode 22 sufficiently large such that the rate of heat deposited in zone 32 is less than or equal to the rate at which the body removes heat from zone 32. Typically, the return electrode 22 is generally made about 10 times to about 100 times as large as the treatment electrode 18 to prevent or minimize heating in zone 32 and to keep any heating in zone 32 below therapeutic amounts.

FIG. 1c is a simplified electrical schematic of the device of FIGS. 1a, 1b and 5. In FIG. 1c, resistor r₁ represents the total resistance or impedance r₁ of the treatment zone 12 (hereinafter referred to as the “local impedance”), and resistor r₂ represents the total resistance or impedance r₂ of the remainder of the electrical circuit associated with the system 10 (hereinafter referred to as the “bulk impedance”). The total system impedance, r₃, is equal to the sum of r₁ and r₂. If the ratio of r₁ to r₂ changes, so will the relative amount of energy absorbed by the treatment zone 12. If this ratio varies in an unknown manner, either too much energy or insufficient energy may be deposited in the treatment zone 12. The ratio of r₁ to r₂ represents a “local impedance factor”, as does the ratio of r₁ to r₃, and the ratio of r₁ to any other portion of the total system impedance. Such ratios are indicative of the fraction or percent of energy delivered by the generator 16 that is being absorbed by the patient treatment zone 12.

To aid in understanding the invention, assume that empirical experiments indicate that optimum therapeutic results are achievable if 50 joules of energy are delivered to a certain treatment zone 12 during an anticipated patient treatment time period (e.g., about 1 second to about 10 seconds) for the delivery of this energy. If the ratio of r₁ to r₂ is 0.5 (i.e., r₁ is one third of total impedance, r₁ plus r₂), the generator should be adjusted to deliver 150 joules of energy. Then 50 joules of energy will be deposited in the treatment zone, as desired. However, if the ratio is less than 0.5, too little energy will be deposited in the treatment zone, and if the ratio is more than 0.5 too much energy will be deposited. The invention seeks to estimate this ratio and to also estimate how r₁ varies relative to other system impedances so that the energy generated by the generator 16 may be adjusted, with the result that desired and appropriate amounts of energy will be deposited in the treatment zone.

The system 10 is operable to determine a local impedance factor for a patient treatment zone 12, which is adjacent a skin surface 14, that is treated by the system 10.

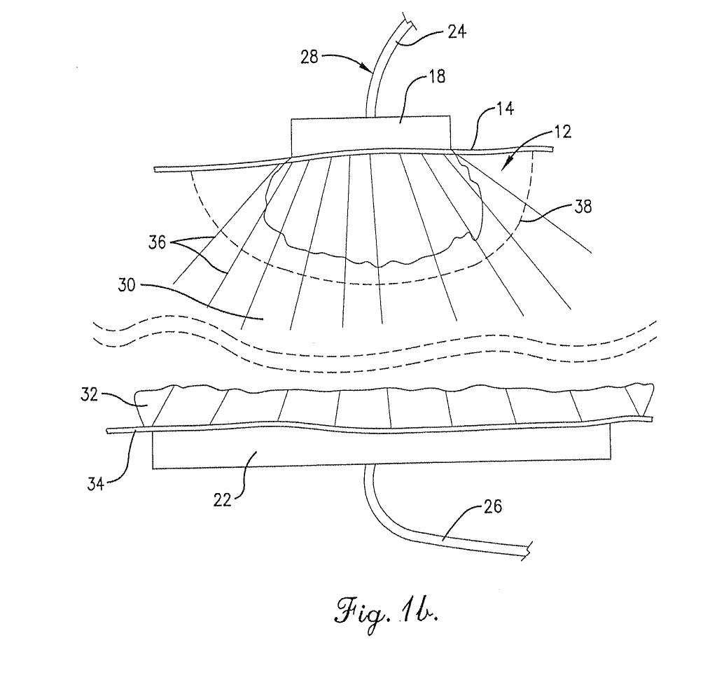

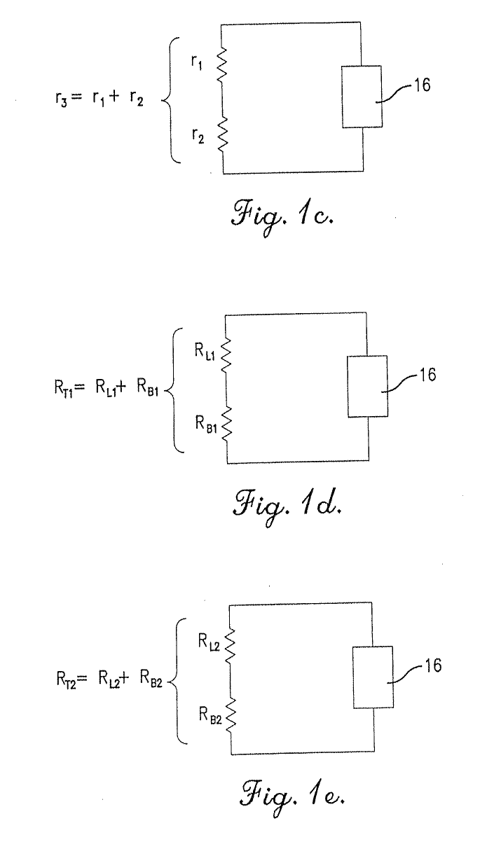

According to a first specific embodiment of the invention, one or a series of first measurement signals are generated by the generator 16 to calculate an approximation of the bulk impedance r₂, and a second measurement signal is generated to calculate an approximation of the total system impedance r₃. Given approximations of r₂ and r₃, r₁ may be readily estimated as may various local impedance factors. r₃ may be approximated by measuring a total system impedance with the treatment electrode 18 in place. This is accomplished by sending a generator measurement signal along cable 24, and measuring any combinations of currents, voltages and impedances associated with the measurement signal. Then, r₂ may be approximated by replacing the treatment electrode 18 with a large area electrode 48 (FIG. 2) sized sufficiently large such that a local impedance in the vicinity of this electrode is minimal and, optimally, is near zero. For example, if the large area electrode 48 is the same order of magnitude in size as the return electrode 22 (which is generally made sufficiently large so that therapeutic heating does not occur in its vicinity), r₁ should be minimal.

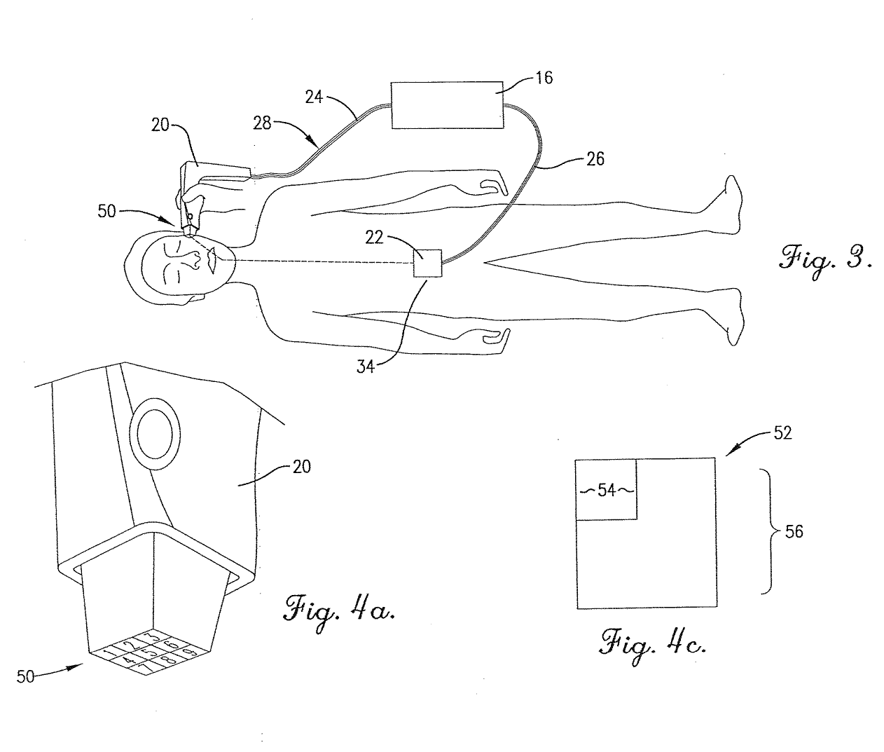

According to a second specific embodiment of the invention and with reference to FIG. 4a, r₃ may be measured, as described above, using a measurement electrode 50 in the form of a multiplexed structure having a 3 by 3 array of individual electrode segments, which are labeled with the numbers 1-9. A series of measurement signals then may be applied to different groups of the individual electrode segments of electrode 50 to define energized electrode blocks of progressively increasing area, for example area or individual electrode 1; followed by areas 1, 2, 4 and 5; and then areas 1-9. Alternatively, the series of measurement signals may also be made by energizing all electrode segments in the array simultaneously, then a group of adjacent electrode segmetns in the array simultaneously, and then each of the electrode segments in the array individually.

Impedances are measured for each measurement signal of this series. If the size of the array is smaller than the return electrode 22, then these impedances may be used in a curve fit to extrapolate to an approximate large size electrode 48 to provide the bulk impedance r₂. FIG. 4b shows three such measurement impedances 201-203, and how these impedances 201-203 may be extrapolated to estimate the bulk impedance. Better estimations can be achieved by using arrays having larger numbers of electrodes, for example 4 by 4, 5 by 5, 6 by 6 , . . . , 100 by 100, and even larger, and by using correspondingly larger numbers of measurement signals associated with this series.

For each of these specific embodiments of the invention, two assumptions are made that may bear on the exact implementation of these impedance estimates. The first assumption is that using two large electrodes will provide an accurate bulk impedance estimate that has no significant local impedance component. The second assumption is that the local impedance is only local and contains none of the bulk impedance component. It is possible that one of these assumptions may be incorrect for certain types of treatment electrodes 18 and/or for certain areas of a body being treated. One or more scaling factors may be empirically derived and used to compensate for inaccuracies introduced by these assumptions. Empirical measurements may result in a finding of a single scaling factor useful for all treatment electrodes, or perhaps different scaling factors for different treatment electrodes. Typical scaling factors and their relation to total, bulk and local impedances are given by:
Z_{L}=(Z_{T}−K_{B}Z_{B})/K_{L}
where Z_{L} is the local impedance, Z_{T} is the total impedance, Z_{B} is the bulk impedance, K_{B} is an empirically derived bulk constant, and K_{L} is an empirically derived bulk constant.

With reference to FIG. 4c and according to a third specific embodiment of the invention, a patient treatment electrode 52 may be used to acquire the measurement impedances from which r₁, r₂ and r₃ are estimated, as opposed to a large area electrode 48 (FIG. 2) that is not actually used for patient treatment. As in the previous embodiment, the treatment electrode 52 includes an array of at least two electrode segments 54, 56. Each individual electrode segment 54, 56 has a different cross sectional area size. For example, referring to FIG. 4c, treatment electrode 52 may be constructed such that electrode segment 54 has an area that is a quarter of the area of electrode segment 56, i.e., the area 54 is a subset of the area 56.

A first measurement signal is sent through the first electrode segment 54 of the array represented by treatment electrode 52 and an impedance measured. Then, a second measurement signal is sent through another electrode segment 56 of the array and a second impedance is measured. So long as the area of the first electrode segment 54 differs from the area of the second electrode segment 56, algorithms know to a person having ordinary skill in the art may be used to estimate local, bulk, and total impedance from these two measurements. It may readily be appreciated the electrode 50 of FIG. 4c could similarly be used as both a treatment and measurement electrode.

In an exemplary algorithm, the two measurement electrode segments 54, 56 are used for obtaining the measurement signals and the second electrode segment 56 corresponds to the electrode area that will be used to deliver therapeutic energy or a treatment signal after the local impedance factor estimate is made. A measurement signal is sent through electrode segment 54, and a first total resistance r_{T1} is measured. Then, a second measurement signal is sent through the electrode segment 56 and a second total resistance r_{T2} is measured. Electrical schematics of these two measurement signals are shown in FIGS. 1d, 1e, where the subscripts L and B represent local and bulk respectively, and otherwise these Figures use nomenclature consistent with FIG. 1c. If the electrode segment 54, 56 have respective areas Aₗ, and A₂ respectively, and if it is assumed the treatment depths of the two electrode areas 54, 56 are identical, the tissue resistivity of the respective treatment zones are the same, and the bulk impedances are the same for each electrode, the local impedance is given by:
r_{L1}=(r_{T1}−r_{T2})/(1−(A₁/A₂))
and
r_{L2}=r_{T2}+r_{L1}−r_{T1}

Because the total system impedance is represented by r_{T2}, the desired local impedance factors may be readily calculated and estimated.

As with the prior two embodiments, empirically derived scaling factors may be determined to compensate for inaccuracies introduced by various assumptions used above.

Algorithms may be incorporated into the system 10 for computing the local impedance value and the fraction of total impedance which is in the patient treatment zone 12. This process may result in the ability to predict a safer treatment range for each patient. Patients may be treated under deeper anesthesia, thus eliminating patient discomfort during treatment, after safe and effective treatment settings are forecasted and estimated by the system for each patient.

Relays, switches, and other controllable elements may be coupled with measurement electrode 50 to selectively energize various electrode areas 1-9 and with measurement electrode 52 to selectively energize electrode segments 54, 56, as described above. For example, the controller 44 may be coupled with the relays to select various relays to enable the propagation of energy through selected electrode areas 1-9 of measurement electrode 50, or electrode segments 54, 56 of measurement electrode 52. The various control elements may be integral with the electrodes 50, 52, the handpiece 20, the generator 16, and/or other system 10 elements. The system 10 may additionally include other elements, such as conventional computing elements and/or data storage elements. For example, the conventional computing elements and data storage elements may enable the system 10 to record, store, track, and analyze various data sensed by the sensors or otherwise inputted into the system 10. Furthermore, in some embodiments, the treatment electrodes 18, 50, 52 and/or handpiece 20 may include data storage elements, such as an EPROM, to store specific data regarding the particular electrodes being utilized. Thus, data corresponding to the treatment of the patient, such as previous treatments of the patient, determined patient impedance factors, etc, may be stored and recalled later by the computing elements or controller 44 for use during treatment. Additionally, the generator 16 may be operable to utilize stored data to estimate local, bulk, and total impedance factors for the patient based upon previously stored data.

Throughout treatment of the patient, measurement of local, bulk and/or total system impedance may be repeated to continually determine the local impedance factors associated with each treatment zone. For example, the system 10 may be utilized to continually determine the local impedance factor during treatment of the patient through use of the treatment electrodes 50, 52. So, for example, if a patient's full face is being treated by an RF treatment tip having a three cm² area, it would be typical to deliver a therapeutic energy or treatment signal to the patient repetitively, say 100, 200, 300 or as much as 600 times as different areas of the face are heated. The local impedance factor could be repetitively determined, and the energy delivered repetitively varied in response to the local impedance factors so determined.

System 10 may be used for any therapeutic, medical, and/or cosmetic-related treatment. For example, the system 10 may be a radiofrequency, microwave, ultrasound, infrared, optical, laser, acoustic, electromagnetic, or other similar energy generating device. Such energy-based systems, including the system 10, generally direct energy at a patient to heat tissue and modify various patient physical properties, such as tissue appearance, physical tissue structure, etc. In particular, system 10 may be a radiofrequency based system, such as the ThermaCool® systems commercially available from Thermage Inc. (Hayward, Calif.), modified to estimate the local impedance factor for energy delivery as disclosed herein.

Local impedance factors may be estimated using electrode assemblies disclosed in application Ser. No. 11/423,068, filed on Jun. 8, 2006 and entitled “Treatment Apparatus and Methods for Delivering Energy at Multiple Selectable Depths in Tissue”; the disclosure of the referenced application is hereby incorporated by reference herein in its entirety.

While the invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicants' general inventive concept.

What is claimed is:

1. A method of treating a treatment zone of a patient with an electromagnetic energy delivery device, the method comprising:
sending a first measurement signal from the electromagnetic energy delivery device at least partially through the treatment zone and back to the electromagnetic energy delivery device;
determining a first measurement value from the first measurement signal;
sending a second measurement signal from the electromagnetic energy delivery device at least partially through the treatment zone and back to the electromagnetic energy delivery device;
determining a second measurement value from the second measurement signal; and
estimating a local impedance factor associated with the treatment zone using the first and second measurement values.

2. The method of claim 1 wherein determining the first measurement value further comprises:
measuring a current or a voltage associated with the first measurement signal.

3. The method of claim 1 wherein determining the second measurement value further comprises:
measuring a current or a voltage associated with the second measurement signal.

4. The method of claim 1 wherein estimating the local impedance factor further comprises:
determining the local impedance factor as a ratio between a patient local impedance associated with the treatment zone and a total system impedance of the device.

5. The method of claim 1 further comprising:
selecting an energy of a therapeutic signal at least partially based upon the estimated local impedance factor; and
sending the therapeutic signal from the electromagnetic energy delivery device to the treatment zone.

6. The method of claim 5 further comprising:
repeatedly estimating local impedance factors during the course of the patient treatment.

7. The method of claim 6 further comprising:
changing the energy of the therapeutic signal sent to the treatment zone as the estimated local impedance factor changes during the course of the patient treatment.

8. The method of claim 5 wherein the local impedance factor is related to a fraction of the energy of the therapeutic signal absorbed by the patient treatment zone.

9. The method of claim 5 further comprising:
sending the first measurement signal, the second measurement signal, and the therapeutic signal through different electrodes located adjacent the treatment zone.

10. The method of claim 9 wherein an area of the electrode used for sending the second measurement signal is equal to the area of the electrode used for sending the therapeutic signal.

11. The method of claim 9 wherein an area of the electrode used for sending the second measurement signal differs from the area of the electrode used for sending the therapeutic signal.

12. The method of claim 5 further comprising:
sending the first measurement signal, the second measurement signal, and the therapeutic signal through a plurality of individual electrodes each located adjacent the treatment zone.

13. The method of claim 5 wherein the device delivers radiofrequency energy, and the return path includes a non-therapeutic electrode of sufficient size such that a non-therapeutic amount of energy is delivered to a patient zone adjacent the non-therapeutic electrode.

14. The method of claim 1 further comprising:
sending more than two measurement signals through the treatment zone;
determining a corresponding number of measurement values from the more than two measurement signals; and
estimating the local impedance factor from the corresponding number of measurement values.

15. The method of claim 14 wherein estimating the local impedance factor further comprises:
estimating the local impedance factor by extrapolation.

16. The method of claim 1 wherein estimating the local impedance factor further comprises:
subtracting the second measurement value from the first measurement value to yield a difference; and
dividing the difference by one minus a ratio of a surface area of a first electrode used to send the first measurement signal to a surface area of a second electrode surface used to send the second measurement signal.

17. The method of claim 1 wherein estimating the local impedance factor further comprises:
using one or more scaling factors to estimate the local impedance factor.

18. The method of claim 1 further comprising:
beginning a patient treatment session after the local impedance factor is estimated.

19. The method of claim 1 further comprising:
repetitively sending respective therapeutic signals through individual treatment zones; and
estimating local impedance factors associated with each of the treatment zones.

20. The method of claim 19 further comprising:
changing an energy of the therapeutic signal as the estimated local impedance factor changes.

21. The method of claim 1 wherein one of the first and second measurement values is approximately equal to a total impedance of the electromagnetic energy delivery device.

22. The method of claim 1 wherein one of the first and second measurement values is approximately equal to a bulk impedance of the electromagnetic energy delivery device.

23. An apparatus for deliver electromagnetic energy through a skin surface to an underlying treatment zone of a patient, the apparatus comprising:
a generator adapted to generate the electromagnetic energy;
a treatment tip including an electrode operatively coupled with said generator to deliver the electromagnetic energy through the skin surface and into the patient treatment zone; and
a controller electrically coupled with the generator, the controller configured to cause the generator to supply at least first and second measurement signals to the electrode for delivery to the treatment zone, and the controller configured to estimate a local impedance factor of the patient treatment zone from the first and second measurement signals.

24. The apparatus of claim 23 wherein the generator is adapted to generate radiofrequency energy.

25. The apparatus of claim 23 wherein the electrode further comprises a first and second electrode segments having different surface areas, and the controller is configured to deliver the first measurement signal through the first electrode and the second measurement signal through the second electrode.

26. The apparatus of claim 23 wherein the electrode includes a plurality of electrode segments, and and the controller is configured to deliver the first measurement signal through a first group of the electrode segments and the second measurement signal through a second group of the electrode segments, the first and second groups of electrode segments having a different collective surface areas.

27. The apparatus of claim 23 wherein the controller is configured to cause the generator to supply a therapeutic signal to the electrode based upon the local impedance factor estimated by the controller.
